(19) United States
(12) Patent Application Publication
(10) Pub. No.: US 2026/0066085 A1
(43) Pub. Date: Mar. 5, 2026

(54) System and Method for AI-Driven Surgical Care Recommendation and Optimization

(71) Applicant: Juliette Koval, San Francisco, CA (US)

(72) Inventor: Juliette Koval, San Francisco, CA (US)

(21) Appl. No.: 18/816,274

(22) Filed: Aug. 27, 2024

(51) Int. Cl. G16H 20/40; G06F 16/9535; G16H 20/10; G16H 20/30

(52) U.S. Cl. CPC G16H 20/40; G06F 16/9535; G16H 20/10; G16H 20/30

(57) ABSTRACT

A system and method for recommending surgical care providers and protocols using artificial intelligence (AI) and machine learning (ML) is disclosed. The system comprises one or more processors coupled to a memory that stores instructions for receiving healthcare data from various sources, preprocessing the data, training an ML model to generate recommendations, and iteratively refining the model based on patient feedback. The system receives data related to surgical procedures, hospitals, doctors, and pre- and post-operative care, preprocesses the data using techniques such as data cleaning, normalization, and encoding, and trains a neural network model to generate recommendations for selecting optimal hospitals, doctors, and care protocols. The generated recommendations are provided to users via an intuitive interface, and patient feedback is used to retrain and optimize the ML model for continuous improvement.

### BACKGROUND OF THE INVENTION

### Field of Invention

The various aspects discussed herein relate to a system and method for recommending surgical care providers and protocols using artificial intelligence and machine learning.

### Description of Related Art

Patients undergoing surgical procedures face challenges in selecting the most suitable hospitals and doctors for their specific needs. Additionally, there is a lack of personalized guidance regarding optimal preoperative and postoperative care protocols. Existing systems for recommending surgical care providers often fail to leverage the full potential of available healthcare data and advanced analytical techniques. Consequently, patients may not receive the highest quality care, leading to suboptimal outcomes and patient experiences.

Accordingly, there is a need in the art for an intelligent system and method that can comprehensively analyze healthcare data from multiple sources, provide data-driven recommendations for selecting hospitals and doctors best suited for particular surgeries, and offer evidence-based, personalized preoperative and postoperative care protocols to optimize patient outcomes and satisfaction. By harnessing artificial intelligence and machine learning technologies to process vast amounts of surgical care data, such a system can enable patients to make more informed decisions and receive higher quality, tailored care throughout their surgical journey.

### BRIEF SUMMARY OF THE INVENTION

This summary is provided to introduce a selection of concepts, in a simplified format, that are further described in the detailed description of the invention. This summary is neither intended to identify key or essential inventive concepts of the invention nor is it intended for determining the scope of the invention.

The present invention relates to a system and method for recommending surgical care providers and protocols using artificial intelligence (AI) and machine learning (ML). In one embodiment, the system comprises one or more processors coupled to a memory that stores instructions for receiving healthcare data from various sources, preprocessing the data, training an ML model to generate recommendations, and iteratively refining the model based on patient feedback.

The system receives healthcare data related to surgical procedures, hospitals, doctors, and pre- and post-operative care from sources such as federal healthcare databases, hospital electronic health records (EHRs), and patient surveys. The data is preprocessed using techniques like data cleaning, normalization, and encoding to ensure quality and consistency.

A neural network model, a deep learning architecture with multiple hidden layers, is trained on the preprocessed data using supervised learning. The model generates recommendations for selecting the most suitable hospitals and doctors for a given surgical procedure based on factors such as outcomes, complication rates, patient satisfaction, and doctor experience. It also determines optimal preoperative and postoperative protocols, including patient education, dietary restrictions, medication regimens, wound care, and physical therapy.

The generated recommendations are provided to users via an intuitive interface, such as a web-based dashboard with search functionality and data visualizations. Patients can offer feedback on the recommendations, which is then used to retrain and optimize the ML model for continuous improvement.

Advantageously, the present invention leverages AI and ML to comprehensively analyze vast amounts of surgical care data, empowering patients to make data-driven decisions and receive personalized, evidence-based care. By iteratively refining its recommendations based on real-world patient feedback, the system continuously improves its performance and adapts to evolving patient needs and preferences.

Additional features and advantages of the invention will be set forth in the description which follows. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a block diagram illustrating a system for recommending surgical care providers and protocols, according to an embodiment of the invention

FIG. 2 illustrates a user interface diagram for interacting with the surgical care recommendation system, according to an embodiment of the invention.

FIG. 3 is a system flow diagram illustrating the end-to-end user interaction and backend processes of the surgical care recommendation system, according to an embodiment of the invention.

FIG. 4 is another system flow diagram illustrating the end-to-end user interaction and backend processes of the surgical care recommendation system, according to an embodiment of the invention.

FIG. 5A illustrates a user profile.

FIG. 5B illustrates hospital data.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

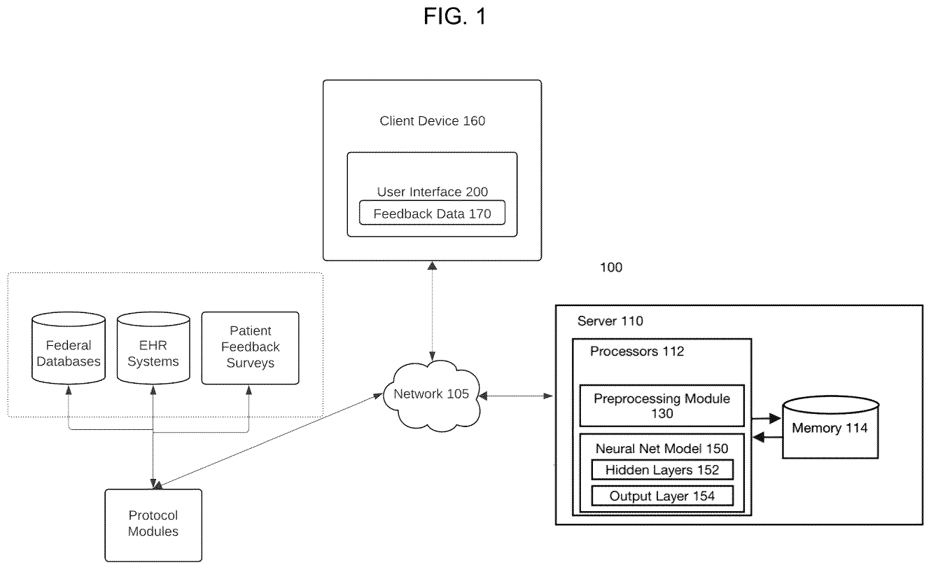

FIG. 1 is a block diagram illustrating a system (100) for recommending surgical care providers and protocols according to an embodiment of the invention. The system (100) comprises a server (110) that may include one or more processors (112) coupled to a memory (114). The memory (114) can store instructions that, when executed by the processor(s) (112), cause the system (100) to perform operations including but not limited to:
(a) receive healthcare data from a plurality of sources (120) over a network (105), wherein the healthcare data is related to surgical procedures, hospitals, doctors, and pre- and post-operative protocols;
(b) preprocess the received healthcare data using a data preprocessing module (130) to ensure data quality, wherein said preprocessing may include applying data cleaning techniques to handle missing or inconsistent data, normalizing data formats across different sources, and encoding categorical variables using techniques such as, by way of example and not limitation, one-hot encoding;
(c) train a neural network model (150) using the preprocessed healthcare data to generate recommendations for selecting hospitals and doctors for particular surgical procedures, wherein the neural network model can have a deep learning architecture with a plurality of hidden layers (152) and an output layer (154), and is trained using a supervised learning approach with labeled training data; and
(d) train the neural network model (150) using the preprocessed healthcare data to generate recommendations for preoperative and postoperative protocols for particular surgical procedures.

As shown in FIG. 1, the server (110) is configured to receive healthcare data from a plurality of sources (120) over a network (105). The network (105) may be a wide area network (WAN), such as the Internet, a local area network (LAN), or any other suitable type of network. The sources (120) can include, but are not limited to:
Federal healthcare databases (122) providing data on surgical outcomes, complications, and costs across different providers and geographies, such as databases maintained by the Centers for Medicare & Medicaid Services (CMS) or the Agency for Healthcare Research and Quality (AHRQ);
Hospital electronic health record (EHR) systems (124) containing detailed patient data, surgical details, and treatment outcomes, which may be accessed via application programming interfaces (APIs) or health information exchanges (HIEs) in compliance with healthcare data privacy and security regulations such as the Health Insurance Portability and Accountability Act (HIPAA);
Patient feedback surveys (126) capturing subjective ratings and comments on care experiences and satisfaction levels, which can be collected via web-based forms, mobile apps, or interactive voice response (IVR) systems.

The received healthcare data generally covers various aspects of surgical care, including details on surgical procedures, hospitals, doctors, and pre- and post-operative protocols. The data may be structured, semi-structured, or unstructured, and can be in various formats such as, by way of example and not limitation, comma-separated values (CSV), extensible markup language (XML), or JavaScript object notation (JSON).

Upon receiving the healthcare data, the server (110) preprocesses the data using the data preprocessing module (130) to ensure high data quality and consistency. In one embodiment, the preprocessing steps may include applying data cleaning techniques to handle missing or inconsistent values, such as imputation or removal of incomplete records; normalizing data formats across the plurality of sources (120), such as converting all date fields to a standard format; and encoding categorical variables using techniques like one-hot encoding to convert them into numerical representations suitable for machine learning. The preprocessed data can then be stored in a database (140), which may be a relational database management system (RDBMS) such as MySQL or PostgreSQL, or a NoSQL database such as MongoDB or Cassandra, for further use.

With reference to FIG. 1, the server (110) is further configured to train the neural network model (150) using the preprocessed healthcare data from the database (140). The neural network model (150) can have a deep learning architecture with multiple hidden layers (152) and an output layer (154) for generating recommendations. The hidden layers (152) may use various types of neural network units, such as fully connected layers, convolutional layers, or recurrent layers, depending on the nature of the input data and the desired output. The model training process typically employs a supervised learning approach, using labeled training examples from the database (140), where each example consists of input features (e.g., patient characteristics, surgical procedure details) and corresponding output labels (e.g. risk factors, likelihood of complications, recommended protocols). Model parameters, such as connection weights between neurons, can be optimized using a gradient descent algorithm, such as stochastic gradient descent (SGD) or Adam, to minimize a loss function that quantifies the difference between predicted and actual outputs. This dataset, pulled from multiple sources (CMS, EHR, Patient Feedback), creates base weights for each feature for each type of surgery. The base weights are then adjusted (re-weighted) when the user inputs their profile to enhance the recommendations accounting for their individual risk factors. Regularization techniques, such as L1/L2 regularization or dropout, may be applied to prevent overfitting and improve generalization to unseen data.

In one embodiment, the neural network model (150) is trained to generate two types of recommendations, including but not limited to:
1. Recommendations for selecting the most suitable hospitals and doctors for a particular surgical procedure, based on factors including but not limited to surgical outcomes (e.g., mortality rates, complication rates), patient satisfaction scores (e.g., Hospital Consumer Assessment of Healthcare Providers and Systems (HCAHPS) scores), and doctor experience level (e.g., number of procedures performed, years of practice). The model may use techniques such as collaborative filtering or content-based filtering to generate personalized recommendations based on patient preferences and similarities.
2. Recommendations for optimal preoperative and postoperative protocols for a given surgical procedure, including suggested patient education materials (e.g., videos, brochures), dietary restrictions (e.g., fasting requirements, nutritional supplements), medication regimens (e.g., pain management, antibiotic prophylaxis), wound care instructions (e.g., dressing changes, suture/staple removal), and physical therapy exercises (e.g., range of motion, strengthening). The model may use techniques such as rule-based reasoning or decision trees to generate recommendations based on clinical guidelines and best practices.

Once trained, the neural network model (150) can be deployed on the server (110) and used to provide personalized surgical care recommendations to users via a client device (160). The client device (160) may be a personal computer, laptop, tablet, smartphone, or any other computing device capable of running a web browser or a dedicated application. As illustrated in FIG. 1, the client device (160) includes a user interface (200), such as a web-based dashboard or a mobile app, that allows users to input their specific surgical requirements and preferences, such as the type of procedure, desired location, insurance coverage, Social Determinants of Health, and medical history. The user interface (200) communicates with the server (110) over the network (105) using standard protocols such as hypertext transfer protocol (HTTP) or secure HTTP (HTTPS) to retrieve the relevant recommendations generated by the neural network model (150).

To continuously improve the accuracy and relevance of its recommendations, the system (100) also includes a feedback loop. After receiving care based on the system's recommendations, patients can provide feedback data (170) via the user interface (200). This feedback data (170) generally comprises subjective ratings of their care experience and quality of care, objective health outcomes as well as recovery tracking metrics (e.g., pain levels, mobility, wound healing), and suggestions for enhancing the recommendations. The feedback data (170) may be collected using various methods, such as online surveys, mobile apps, or wearable devices that monitor patient recovery progress.

The server (110) collects this patient feedback data (170) and uses it to retrain and refine the neural network model (150). The feedback data (170) is incorporated into the training dataset stored in the database (140), and the model parameters and hyperparameters can be adjusted accordingly using techniques such as transfer learning or fine-tuning. By leveraging this real-world feedback, the system (100) can iteratively update its recommendations to better meet patient needs and preferences, thereby improving patient outcomes and satisfaction over time. The retraining process may be performed periodically (e.g., weekly, monthly, quarterly) or triggered by specific events (e.g., receiving a certain number of new feedback entries).

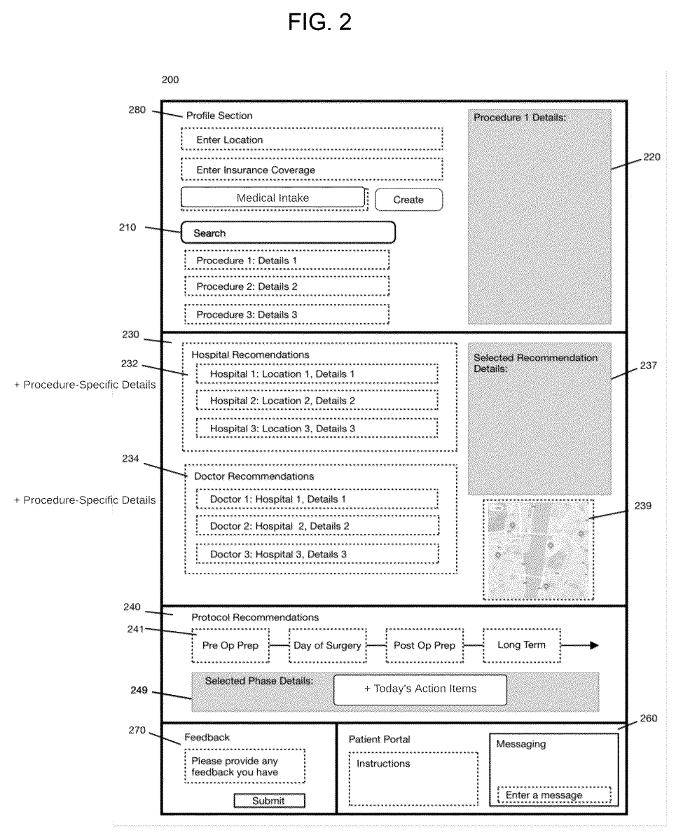

FIG. 2 illustrates a user interface diagram for interacting with the surgical care recommendation system (100) according to an embodiment of the invention. The user interface (200) is displayed on the client device (160) and communicates with the server (110) over the network (105) to provide users with access to personalized surgical care recommendations.

As shown in FIG. 2, the user interface (200) includes a user profile section (280) where users can create an account, input their personal health information, and set preferences for factors such as, but not limited to, location, insurance coverage, and communication methods. This information is securely stored in the database (140) and used by the neural network model (150) to generate personalized recommendations.

In one embodiment, the user interface (200) comprises a procedure search bar (210) where users may enter the name or description of a surgical procedure they are interested in. In some embodiments, the search bar (210) includes an auto-complete feature that suggests relevant procedure names based on the user's input, drawing from the list of procedures stored in the database (140).

Upon selecting a procedure, the user is presented with a procedure details section (220) that generally displays key information about the chosen procedure, wherein said information may comprise a brief description, typical indications, and expected outcomes. This information is derived from the preprocessed healthcare data stored in the database (140).

With reference to FIG. 2, proximate to the procedure details, the user interface (200) displays two main recommendation sections: a hospital and doctor recommendation section (230) and a pre- and post-operative protocol recommendation section (240). These sections present the personalized recommendations generated by the neural network model (150) based on the selected procedure and the user's preferences.

The hospital and doctor recommendation section (230) includes a ranked list of recommended hospitals (232) and doctors (234) for the selected procedure. Each recommendation may comprise the hospital name, location, and key performance metrics such as, but not limited to, surgical volume, complication rates, and patient satisfaction scores. Similarly, each doctor recommendation can include the doctor's name, affiliation, specialty, and experience level. Users can optionally click on a hospital or doctor to view more detailed information and patient reviews in the selected recommendations section (237).

In another embodiment, the hospital and doctor recommendation section (230) presents the recommended providers using interactive maps (239) that substantially show the location and proximity of each hospital, as well as comparison tables (not shown) that allow users to easily assess the key performance metrics and patient reviews for each provider. Users might filter and sort the recommendations based on their preferred criteria.

As illustrated in FIG. 2, the pre- and post-operative protocol recommendation section (240) provides a timeline view of suggested care protocols for the period before and after the surgery. The timeline is generally divided into phases (241) such as, by way of example and not limitation, pre-operative preparation, day of surgery, post-operative recovery, and long-term follow-up. Users may click on a phase (241) to view more detailed information in the selected phase details section (249). Each phase typically includes specific recommendations for patient education, medication regimens, diet and activity restrictions, and rehabilitation exercises. These recommendations are based on the optimal protocols identified by the neural network model (150) for the selected procedure.

With reference to FIG. 2, the patient portal section (260) provides users with access to their personalized pre- and post-operative care instructions. In some embodiments (not shown), the instructions may be presented using interactive timelines that guide users through each phase of the care journey, with embedded video tutorials, printable checklists, and reminders for key milestones. The portal also integrates with the hospital EHR systems (124) to auto-populate relevant patient information and enable secure messaging with the care team, thereby streamlining the user experience and ensuring continuity of care.

The user interface (200) includes a feedback submission form (270) where users can rate their experience with the recommended hospitals, doctors, and care protocols. This feedback is collected and processed by the server (110) to continuously improve the accuracy and relevance of the recommendations generated by the neural network model (150).

Finally, as depicted in FIG. 2, users can customize the recommendations by adjusting inputs such as location, insurance coverage, and personal health factors in the profile section (280). As users interact with the recommendations, their feedback and preferences are captured and sent back to the server (110) as feedback data (170) to refine future recommendations, thereby continuously improving the system's performance.

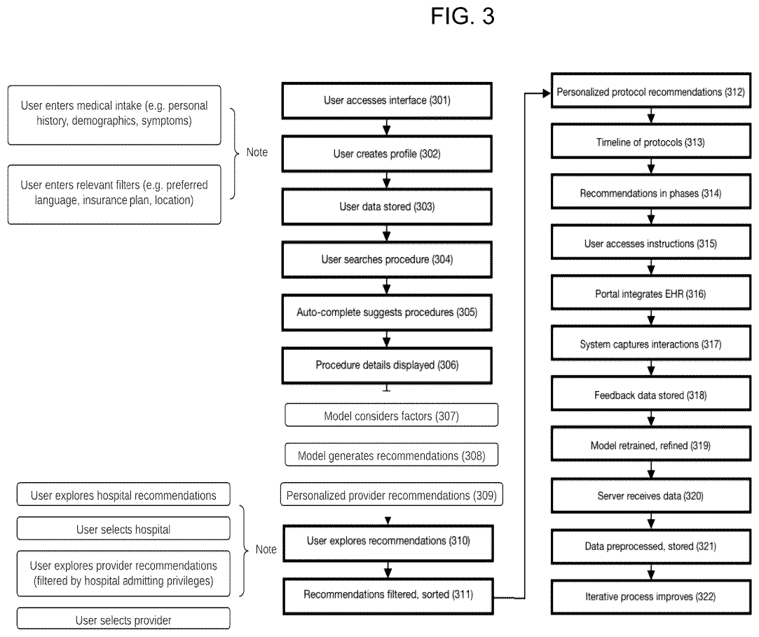

FIG. 3 is a system flow diagram illustrating the end-to-end user interaction and backend processes of the surgical care recommendation system (100) according to an embodiment of the invention.

In one embodiment, the user flow may begin with the user (301) accessing the system's user interface (200) on their client device (160). The user can create an account and input their personal health information and preferences via the user profile section (280) (302), wherein this information is generally securely transmitted over the network (105) to the server (110) for storage in the database (140) (303).

As shown in FIG. 3, the user may then proceed to search for a specific surgical procedure using the procedure search bar (210) (304). As the user types, the system can provide auto-complete suggestions based on the procedure data stored in the database (140) (305). Upon selecting a procedure, the user interface may display relevant procedure details (220) retrieved from the preprocessed healthcare data (306).

In another embodiment, the user might be presented with personalized recommendations for hospitals (232) and doctors (234) substantially best suited for the selected procedure (307). These recommendations are typically generated by the neural network model (150), which has been trained on the preprocessed healthcare data from various sources (120), including but not limited to federal databases (122), hospital EHR systems (124), and patient surveys (126) (308), wherein the model generally takes into account factors such as surgical outcomes, complication rates, patient satisfaction scores, and doctor experience levels to rank the recommendations (309).

As depicted in FIG. 3, the user can explore the recommended hospitals and doctors using interactive maps, comparison tables, and detailed information pages (310), wherein they may filter and sort the recommendations based on their profile information (280), such as location and insurance coverage, by way of example and not limitation (311).

Simultaneously, as illustrated in FIG. 3, the system may display personalized recommendations for pre- and post-operative care protocols (240) for the selected procedure (312). These recommendations, also generally generated by the neural network model (150), can provide a timeline view of suggested patient education, medication regimens, diet and activity restrictions, and rehabilitation exercises (313), wherein the recommendations are often divided into phases (241), including but not limited to pre-operative preparation, day of surgery, post-operative recovery, and long-term follow-up (314).

In an alternative embodiment, the user might access their personalized care instructions and communicate with their care team through the patient portal (260) (315), wherein the portal is typically coupled to hospital EHR systems (124) to ensure substantially seamless access to relevant medical information and enable secure messaging (316).

Throughout the user journey, as shown in FIG. 3, the system may capture user interactions, preferences, and feedback (270) via the user interface (200) (317), wherein this feedback data (170) is generally transmitted back to the server (110) and stored in the database (140) (318). The neural network model (150) can be periodically retrained using this real-world feedback data to refine and improve future recommendations (319), thereby potentially enhancing the system's performance.

On the back end, the server (110) may continuously receive healthcare data from various sources (120), such as federal databases (122), hospital EHR systems (124), and patient surveys (126), by way of example and not limitation (320). The data preprocessing module (130) can clean, normalize, and encode this data before storing it in the database (140) for use in training and updating the neural network model (150) (321).

This iterative process, comprising data ingestion, preprocessing, model training, user interaction, and feedback incorporation, can enable the surgical care recommendation system (100) to provide increasingly accurate and personalized recommendations to users over time, ultimately improving patient outcomes and satisfaction (322).

The surgical care recommendation engine will be periodically updated (e.g. weekly, monthly, quarterly) or triggered by specific events (e.g., receiving a certain number of outcome data points). By partnering with the patients'insurance provider, information will be extracted at both company-level and, where compliant with HIPAA regulations, procedure-level within that company. The data extracted from these agreements includes average cost of procedure, readmission rate, complication rate, most common complications, frequency of each complication, and average length of stay. This data can then be benchmarked against the company's previous year's metrics, the metrics for members of the company who elected not to use the service, as well as the metrics for similar corporations in the same time period who have not licensed the service. In one embodiment, this will be transmitted via JSON and CSV files meeting the Health Level Seven International (HL7) Clinical Document Architecture standard. Patient surveys will also be used to gauge outcomes.

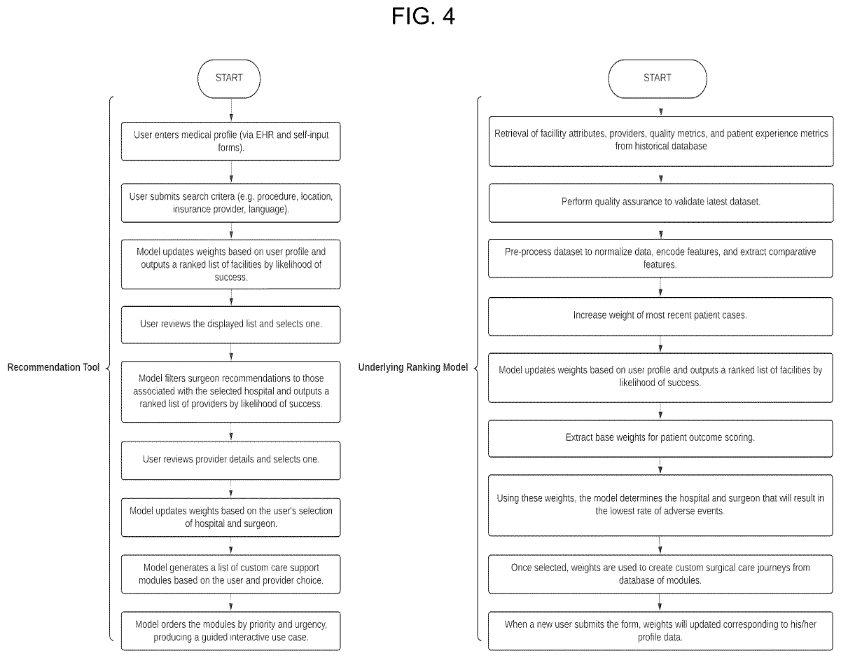

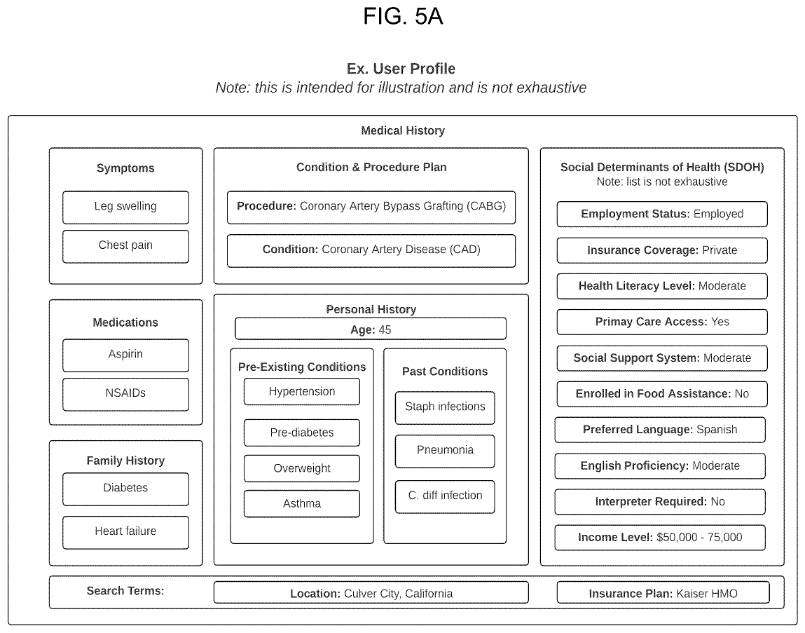

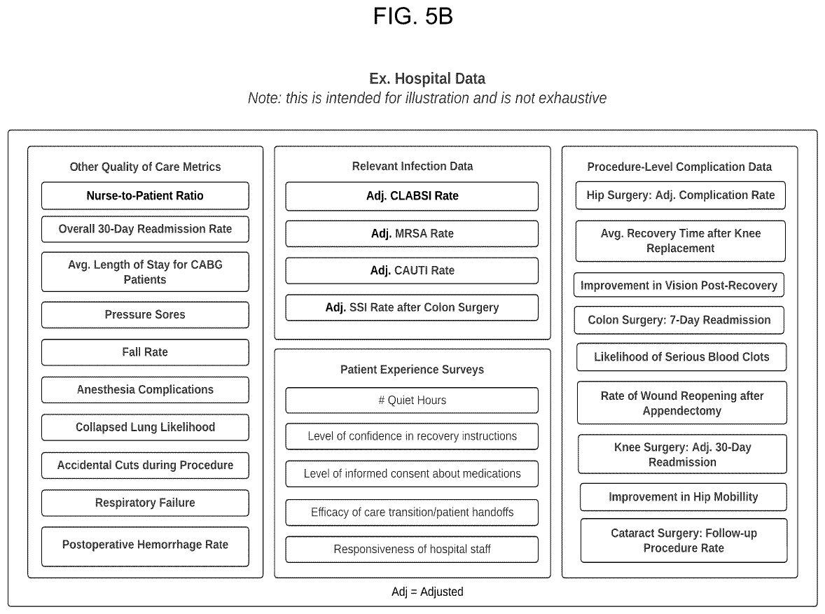

FIG. 4 is another system flow diagram illustrating the end-to-end user interaction and backend processes of the surgical care recommendation system, according to an embodiment of the invention, FIG. 5A illustrates a user profile, and FIG. 5B illustrates hospital data, all to be used in conjunction with the embodiments described.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A system for recommending surgical care providers and protocols, comprising:
a. one or more processors coupled to a memory, the memory storing instructions that, when executed by the one or more processors, cause the system to:
i. receive healthcare data from a plurality of sources, the healthcare data related to surgical procedures, hospitals, doctors, and pre and postoperative protocols;
ii. preprocess the received healthcare data to ensure data quality;
iii. train a neural network model using the preprocessed healthcare data to generate recommendations for selecting hospitals and doctors for particular surgical procedures;
iv. train the neural network model using the preprocessed healthcare data to generate recommendations for preoperative and postoperative protocols for particular surgical procedures;
v. provide the generated recommendations to a user via a user interface;
vi. receive feedback data from patients related to the recommendations; and
vii. retrain the neural network model based on the received feedback data to iteratively refine the recommendations.

2. The system of claim 1, wherein the plurality of sources comprises one or more of: federal healthcare databases, hospital electronic health record systems, and patient feedback surveys.

3. The system of claim 1, wherein preprocessing the received healthcare data comprises: applying data cleaning techniques to handle missing or inconsistent data; normalizing data formats across different sources; and encoding categorical variables using one-hot encoding.

4. The system of claim 1, wherein the neural network model comprises a deep learning architecture with multiple hidden layers and an output layer for generating the recommendations.

5. The system of claim 4, wherein training the neural network model comprises: using a supervised learning approach with labeled training data; optimizing model parameters using a gradient descent algorithm; and applying regularization techniques to prevent overfitting.

6. The system of claim 1, wherein the generated recommendations for selecting hospitals and doctors are based on factors comprising: surgical outcomes, complication rates, patient satisfaction scores, and doctor experience level.

7. The system of claim 1, wherein the generated recommendations for preoperative protocols comprise suggested: pre-surgical patient education materials; dietary restrictions; and medication regimens to optimize surgical outcomes.

8. The system of claim 1, wherein the generated recommendations for postoperative protocols comprise suggested: medication types and dosages to manage pain and prevent complications; wound care instructions; and physical therapy exercises to aid recovery.

9. The system of claim 1, wherein the user interface comprises: a web-based dashboard with search functionality to find recommendations by procedure type; and data visualizations comparing recommended hospitals and doctors based on key performance metrics.

10. The system of claim 1, wherein retraining the neural network model based on the received feedback data comprises:
a. updating the training dataset with new patient feedback examples;
b. adjusting model hyperparameters to improve recommendation accuracy;
c. and periodically re-evaluating model performance on a validation dataset.

11. A method for generating surgical care recommendations using artificial intelligence, comprising:
a. receiving, by one or more processors coupled to a memory, healthcare data from a plurality of sources, the healthcare data related to surgical procedures, hospitals, doctors, and pre and postoperative care;
b. preprocessing, by the one or more processors, the received healthcare data to ensure data quality;
c. training, by the one or more processors, a machine learning model using the preprocessed healthcare data to:
d. rank hospitals and doctors based on their suitability for particular surgical procedures, and
e. determine optimal medication regimens and care protocols for preoperative and postoperative periods for particular surgical procedures;
f. outputting, by the one or more processors, the rankings and determinations as recommendations to a user;
g. receiving, by the one or more processors, feedback data from patients related to the recommendations; and
h. retraining, by the one or more processors, the machine learning model based on the received feedback data to optimize the recommendations.

12. The method of claim 11, wherein preprocessing the received healthcare data comprises:
a. removing duplicate or irrelevant data entries;
b. standardizing data formats across the plurality of sources; and
c. imputing missing values using statistical techniques.

13. The method of claim 11, wherein the machine learning model comprises a deep neural network with:
a. an input layer for receiving the preprocessed healthcare data;
b. a plurality of hidden layers for extracting features and patterns from the data; and
c. an output layer for generating the rankings and determinations.

14. The method of claim 13, wherein the deep neural network further comprises:
a. convolutional layers for processing structured data such as images or time series; and
b. recurrent layers for processing sequential data such as patient histories or treatment timelines.

15. The method of claim 11, wherein ranking hospitals and doctors based on their suitability for particular surgical procedures involves:
a. identifying key performance metrics such as mortality rates, complication rates, and patient satisfaction scores; and
b. calculating weighted scores for each hospital and doctor based on these metrics.
c. adjusting the weights for each feature based on the user's profile features.

16. The method of claim 11, wherein determining optimal medication regimens for preoperative and postoperative periods comprises:
a. analyzing patient outcomes data to identify the most effective antibiotics, dosages, and administration schedules for preventing surgical site infections; and
b. personalizing the regimens based on individual patient characteristics such as age, weight, and comorbidities.

17. The method of claim 11, wherein determining optimal care protocols for preoperative and postoperative periods comprises:
a. identifying best practices for patient preparation and recovery (i.e. anesthesia, surgical techniques, and postoperative monitoring; and
b. adapting the protocols to the specific needs and constraints of different healthcare facilities as well as the risk factors and procedure type of the user.

18. The method of claim 11, further comprising:
a. generating a user interface for presenting the rankings and determinations to patients and healthcare providers;
b. wherein the user interface includes interactive visualizations of the key performance metrics and personalized recommendations.

19. The method of claim 11, wherein the feedback data from patients includes:
a. subjective ratings of the healthcare experience and outcomes;
b. objective measures of health status and recovery progress; and
c. suggestions for improving the recommendations.

20. The method of claim 11, wherein retraining the machine learning model based on the received feedback data involves:
a. using reinforcement learning techniques to update the model parameters and decision rules;
b. with the goal of maximizing long-term patient satisfaction and health outcomes.
